(19) United States
(12) Patent Application Publication
(10) Pub. No.: US 2009/0263436 A1
(43) Pub. Date: Oct. 22, 2009

(54) Material primarily for medical, long-term in vivo use, and method for the production thereof

(76) Inventors: Andreas Kokott, Bad Steben (DE); Bettina Hoffmann, Bayreuth (DE); Gunter Ziegler, Nurnberg (DE); Michael Behr, Regensburg (DE); Martin Rosentritt, Regensburg (DE)

Correspondence Address: JORDAN AND HAMBURG LLP, 122 EAST 42ND STREET, SUITE 4000, NEW YORK, NY, 10168, US

(21) Appl. No.: 11/991,328

(22) PCT Filed: Jul. 27, 2006
(86) PCT No.: PCT/DE2006/001334

§ 371 Date: Feb. 3, 2009

(30) Foreign Application Priority Data
Sep. 1, 2005 (DE) 10 2005 042 078.8

(51) Int. Cl. A61K 9/14; A61K 31/715

(52) U.S. Cl. 424/405; 514/54

(57) ABSTRACT

The invention relates to a material that is used primarily for medical, long-term in vivo purposes, e.g. as a filling material in dentistry. The aim of the invention is to create a material primarily for medical, long-term in vivo use which does not have the disadvantages of materials used in prior art, does not release active substances, and endures after the material has been removed or when the shape thereof is modified. The aim is achieved by the fact that the material is made of polymers and filling agents which are embodied as polymer-coated, chemically modified particles that carry hydroxyl groups and are surrounded by a matrix of another polymer. The coating polymer develops an antimicrobial effect.

### BACKGROUND OF THE INVENTION

The invention relates to a material primarily for long-term medical in vivo use, such as e.g. for a filling material in dentistry, and to a method for its production.

It is known that foreign materials that are used in the body (e.g. in the oral cavity) or on the body (e.g. as a catheter) are exposed to the microorganisms present there or can promote penetration of microorganisms into the body.

In the case of the oral cavity, for instance, these microorganisms can be aerobic or anaerobic mixed flora. Among the bacteria strains that occur most frequently there are the caries-inducing Streptococcus mutans [Hellwig E. et al. Einführung in die Zahnerhaltung (Introduction to Tooth Preservation), Urban & Schwarzenberg Verlag, Munich, 1995] and Streptococcus sanguis, which is a pioneer colonizing bacterium.

Among the materials frequently used in the oral cavity are metals, ceramics, polymers, and even mixed materials, so-called composites [Eichner K., Zahnärztliche Werkstoffe und ihre Verarbeitung (Dental Materials and Their Processing), Volume 1, Volume 2, Hüthig Verlag Heidelberg 1988 and Craig G C, Powers J M. Restorative Dental Materials, 11th eg. Mosby, St. Louis 2002].

Of all known dental materials, the composites that are used for fixing or filling materials have the reputation of particularly promoting bacterial accumulation in the oral cavity [Weitmann R T, Eames W B, Plaque accumulation on composite surfaces after various finishing procedures. J Am Dent Assoc 1975; 91: 101-106; Skorland K R, Sonju T. Effect of sucrose rinses on bacterial colonization on amalgam and composite. Acta Odontol Scand 1982; 40: 193-196, and Svanberg M, et al. Mutans streptococci in plaque from margins of amalgam, composite, and glass-ionmer restorations. J Dent Res 1990; 69; 861-864]. Further complicating matters, composites shrink during polymerization, so that micro-fine gaps can occur in fillings and cement joints between the tooth substance (dentin/enamel) and the composite. Bacteria can successfully colonize this fine gap [Hellwig E et al. Einführung in die Zahnerhaltung (Introduction to Tooth Preservation), Urban & Schwarzenberg Verlag, Munich, 1995].

Since these gap spaces are largely out of reach of teeth brushing and the rinse action of saliva, the bacteria grow undisturbed and in a short period lead to carious lesions. Not only can bacteria colonize materials, they can also use some of the carbon in polymers for their metabolism and thus contribute to breaking down the composites [and Craig G C, Powers J M. Restorative Dental Materials. 11th ed. Mosby, St. Louis].

Thus, bacteria are damaging in two ways: their unimpeded growth leads to caries, and in addition they contribute to the gradual destruction of the material.

The release of active substances from medically applicable materials has been known for several decades. Application sites include blood vessels (active substance release from coated stents to dilate the vessel) or bones (for bone infections, implantation of a polymer ball chain made of polymethylmethacrylate (Septopal® from biometmerck) with the antibiotic Gentamycin (Refobacin® from Merck)).

When artificial hip joints are implanted using “cementing”, an antibiotic is also added to the “cement” (hardening polymer mass) in these procedures.

While the release of active substance from the stents is supposed to prevent restenosis, that is, blocking of the blood vessel, the ball chains are used for an existing infection. In hip implantation the antibiotic is used prophylactically to protect against the occurrence of an infection.

Active substances in the form of mouthwash solutions and toothpastes are used in the oral cavity (Lahdenperä M S, Puska M A, Alander P M, Waltimo T, Vallittu P K. Release of chlorhexidine diglugonate and flexural properties of glass fibre reinforced provisional fixed partial denture polymer. J Mat Sci Mat Med 2004; 15: 1349-1353;

Imazato S. Influence of incorporation of antibacterial monomer on curing behaviour of a dental composite. J Dent 1999, 27: 292-297; Imazato S, Torii M. Incorporation of bacterial inhibitor into resin composite. J Dent Res 1994; 73: 1437-1444 and Addy M, Handley R. The effect of the incorporation of chlorhexidine acetate on some physical properties of polymerized and plasticized acrylics. J Oral Rehabil 1981; 8.155-163].

One of the most common oral antibacterial active substances is chlorhexidine digluconate [Lahdenperä M S, Puska M A, Alander P M, Waltimo T, Vallittu P K. Release of chlorhexidine digluconate and flexural properties of glass fibre reinforced provisional fixed partial denture polymer. J Mat Sci Mat Med 2004; 15: 1349-1353]. When used for more than six weeks there is discoloration of the mucosa and irritation to the sense of taste, which is why it does not make sense to use this for a long-term medication.

Regarding dental amalgams, it is known that the release of volatile components such as e.g. copper in the filling gap makes it more difficult or impossible for microorganisms to survive [Skorland K R, Sonju T. Effect of sucrose rinses on bacterial colonization of amalgam and composite. Acta Odontol Scand 1982; 40: 193-196 and Svanberg M, et al. Mutans streptococci in plaque from margins of amalgam, composite, and glass-ionmer restorations. J Dent Res 1990; 69; 861-864].

For composites, ideas are being discussed in which plaque deposits are to be reduced or even prevented by incorporating releasable bactericidal substances [Imazato S., McCabe J. F. Influence of incorporation of antibacterial monomer on curing behaviour of a dental composite. J Dent 1994, 73: 1641-1645, and Imazato S, Torii M. Incorporation of bacterial inhibitor into resin composite. J Dent Res 1994; 73: 1437-1444].

The disadvantage of all of the foregoing solutions, however, is that many of the substances in question that have an antibiotic effect can have allergic or toxic effects. In addition, with the known materials (e.g. cement or filling) it must be assured that an adequate active substance level is provided for the material's entire residence time in the oral cavity.

In addition to the synthetically produced antibiotics, substances that derive from natural products are also used as antibacterially active substances. Among these are inter alia chitosan and its derivatives.

Documents EP 03298098 B1, EP 0389629 B1, EP 1255576 B1, and EP 1237585 B1 disclose hardenable pastes made of different oxides or phosphates with chitosan as a binding agent, the solubility of chitosan being reduced by the alkaline properties of the oxides. The described application in the dental field relates to root filling materials or, due to the lack of stability against the pH in the oral cavity, merely to temporary filling materials.

Known from Japanese specification 02102165 A is a mass that contains chitosan and hydroxy apatite that cannot, however, be used as a ceramic until it has been sintered. The disadvantage of this solution is that during sintering the organic components acting as binders are pyrolyzed.

Specifications EP 0287105 B1 and EP 1296726 B1 disclose a bone-building implant material, made of a glycose aminoglycane with cationic polymers as matrix substances, into which the filler particles of a bone-like composition are incorporated. Chitosan is a glycoseaminoglycan, but the aforesaid specifications expressly describe a bone replacement material that can be resorbed by the body and that can also be used in the jaw area.

Japanese specification 07157434A describes a proliferation inhibitor for bacteria in the oral cavity that is formed by chitosan and its derivatives.

In addition, known from Japanese specification 10130427 A is depositing metal ions on the amino groups of the chitosan or its derivatives, this system being used with hydroxyl apatite.

Japanese specification 05000930 A discloses a similar material made of chitosan derivatives and stannous fluoride.

Until now chitosan has been used only in conjunction with bioresorbable fillers such as e.g. calcium phosphate and acts as a degradable bone filler or as a temporary tooth fill material. Chitosan is used as a bonding agent due to its solubility, which is a function of its pH.

The disadvantage of all known materials is that they do not have any continuous antimicrobial action for long-term in vivo use.

### SUMMARY OF THE INVENTION

The underlying object of the invention is therefore to provide a material primarily for long-term medical in vivo use that avoids the disadvantages of the prior art and initiates without an active substance release and that continues to exist after the material has worn off or despite changes in the material form. In addition, a method for the production of this material is to be provided.

The essence of the invention is comprised in that a material with a polymer basis is prepared, the material providing during its entire residence time an antimicrobial/antibacterial effect in a medical application in the oral cavity, e.g. as a filling or cement, without having any toxic or allergic effect. This action continues even after the material has worn off or after damage.

The material advantageously comprises fill elements in the form of polymers, copolymers, composites, metals, glass-like compounds, pure ceramics, or mixtures of these materials that is coated with a polymer coating in the form of polysaccharides or derivatives thereof, these polymer coatings having an antimicrobial effect and the coated fill elements being enclosed by a matrix comprising another polymer.

It is particularly preferred when this polysaccharide is chitosan.

In accordance with the invention, the polymer, e.g. in the form of chitosan, is modified by deacetylation such that the deacetylated polymer, e.g. the chitosan, can be coupled to a modified silicon dioxide particle surface (aldehyde groups on the particle surface) and then 3-vinylbenzaldehyde can be coupled to the polymer-coated particles.

This coating with anti-microbial effect can also be chemically modified such that carbon-carbon (double) bonds are introduced that are involved in the chemical reaction (e.g. polymerization) during the hardening process.

Moreover, the additional chemical modification can change the dispersion behavior, immobilize activatable starter molecules (initiators that can be activated e.g. chemically, thermally, or under UV light) on the surface, and immobilize the necessary and additional reaction accelerators or regulators for the chemical reaction (e.g. polymerization) for adjusting the chain length on the surface.

The filler activated in this manner is dispersed in a liquid monomer mixture, e.g. bis-GMA, TEGDMA, UDMA, BPO, campherchinones, or ketones such that the inventive material is obtained.

Inventively coating the polymer particles produces an antibacterial effect that continues for extended periods of time, simultaneously bringing about the bond to the polymer matrix and the associated improved dispersion of the particle powder in the liquid phase.

During the dispersion, the terminal vinyl group of the particles (activated fillers) reacts with the monomers, hardening to create a polymer matrix. The activated filler is thus an integrative component of the inventive material due to the chemical bond.

### DETAILED DESCRIPTION OF THE INVENTION

The invention shall be explained in greater detail in the following using the exemplary embodiment.

### 1. Deacetylation of the Chitosan

The chitosan is deacetylated in accordance with the known method with reflux in hydrochloric acid. In accordance with the prior art, the chitosan deacetylated in this manner is purified using a dialysis method and converted to a solid by freeze-drying.

2. Coupling the Deacetylated Chitosan To Modified Silicon Dioxide Particle Surfaces/Coupling of 3-Vinylbenzaldehyde

The hydroxyl groups of silicon dioxide particles are reacted with 3-aminopropyl-triethoxysilane in a mixture of ethanol/water at 45° C.

After the particles/fill elements have been cleaned by rinsing with ethanol, the amino groups are modified with glutaraldehyde at room temperature, forming a Schiff base, and then they are rinsed with water. What is obtained is a terminal aldehyde group on the silicon dioxide particles/fill elements, and it is reacted at room temperature with an aqueous solution of deacetylated chitosan.

The particle surface/fill element surface that has been modified with chitosan is reacted with 3-vinylbenzaldehyde. The excess amino groups of the chitosan react with the 3-vinylbenzaldehyde, forming a Schiff base. The particles/fill elements are cleaned of non-covalently bonded 3-vinylbenzaldehyde by rinsing multiple times with water and is then dried.

Due to this method, the powder/fill elements possesses/possess on its/their surface covalently bonded chitosan, the amino groups of which are partially chemically modified by the reaction with 3-vinylbenzaldehyde.

For producing the material, the modified powder/fill elements are dispersed in the monomer mixture (e.g. Bis-GMA, TEGDMA, UDMA, BPO, campherchinones, or ketones). The terminal vinyl group of the particles/fill elements reacts with the monomers during the reaction (hardening of the filler) to create a polymer matrix. The activated filler is thus chemically bonded to the polymer and with it forms the inventive material.

### 3. The Evidence of Antibacterial Effect Was Provided Using Bacterial Adhesion Tests

Dynamic-mechanical analyses (DMA) and bending tests are performed to demonstrate the chemical integration of the fill elements on the polymer matrix.

Specimens (e.g. in the form of plates) are produced using the inventive material.

It is possible to use e.g. a material with non-modified powder/fill elements according to the prior art as a reference. The proportions of powder/fill elements in the fill material are 20-30 vol %, as is known.

The specimens are exposed to a suspension of bacteria (e.g. Streptococcus sanguis). The bacteria thus have the opportunity to adhere to the surface of the specimen and grow. After 36 hours the superficial bacteria counts for the inventive material are determined quantitatively using fluorescence and a scanning electron microscope and are compared to the reference bacteria counts.

All of the features depicted in the description and in the following claims can be essential to the invention, both individually and in any combination with one another.

1.-4. (canceled)

5. Material in accordance with claim 11, wherein the polymer coating of the particles comprises chitosan.

6.-7. (canceled)

8. Material in accordance with claim 11, wherein the polymer matrix is formed from monomers selected from bis-GMA, TEGDMA, UDMA, BPO, campherchinones, and ketones.

9. (canceled)

10. Method for producing a material primarily for long term medical in vivo use, comprising the following steps:
reacting 3-aminopropyl-triethoxysilane with hydroxyl group-bearing silicon dioxide particles in a mixture of ethanol/water thereby to form amino groups on the particles;
reacting glutaraldehyde with the amino groups, forming a Schiff base, thereby to form terminal aldehyde groups on the silicon dioxide particles;
coating the particles having terminal aldehyde groups with deacetylated chitosan;
reacting the coated particles with 3-vinylbenzaldehyde thereby to chemically modify the coating; and
dispersing the chitosan-coated, chemically modified particles in a monomer mixture and then polymerizing the monomer mixture thereby to form the polymer matrix.

11. Material primarily for long term medical in vivo use, comprising antimicrobial coated particles embedded in a polymer matrix, the coated particles comprising silicon dioxide particles coated with an antimicrobially active polysaccharide modified to have terminal vinyl groups which bond the coated particles to the polymer matrix.
